(19) United States
(12) Patent Application Publication
(10) Pub. No.: US 2020/0078261 A1
(43) Pub. Date: Mar. 12, 2020

(54) THERAPEUTIC LEG DEVICE

(71) Applicant: The Boulevard Group, LLC, Alpharetta, GA (US)

(72) Inventor: Jim Duvall, Alpharetta, GA (US)

(73) Assignee: The Boulevard Group, LLC, Alpharetta, GA (US)

(21) Appl. No.: 16/126,681

(22) Filed: Sep. 10, 2018

(51) Int. Cl. A61H 23/02; A61F 7/00

(52) U.S. Cl. CPC A61H 23/02; A61F 7/02; A61F 7/007

(57) ABSTRACT

A leg massager has an interior covering, and the interior covering has a central raised section that extends from the exterior of the interior covering and forms a first cavity on an interior side of the interior covering. The leg massager further has an exterior covering, and the exterior covering has a central raised section that extends from the exterior of the exterior covering and forms a second cavity on an interior side of the exterior covering. The leg massager further has one or more heating elements contained within the first and second cavities when the interior covering is coupled to the exterior covering and one or more vibration modules contained within the first and second cavities when the interior covering is coupled to the exterior covering.

### BACKGROUND OF THE DISCLOSURE

Often an individual may need therapy on his/her legs. The therapy may be needed outside a traditional physical therapy organization.

In this regard, the individual may have injured his/her leg. Additionally, there are some diseases that affect the legs, for example restless leg syndrome.

In performing therapy, oftentimes heat can aid the individual's injured or diseased leg. In addition, vibration may also help the injured or diseased leg.

### BRIEF DESCRIPTION OF THE DRAWINGS

The system is described with reference to the accompanying drawings. In the drawings, like reference numbers indicate identical or functionally similar elements. Additionally, the left-most digit(s) of a reference number identifies the drawing in which the reference number first appears.

FIG. 1 is an inward view of an exemplary therapeutic leg device in accordance with an embodiment of the present disclosure.

FIG. 2 is an outward view of the exemplary therapeutic leg device as shown in FIG. 1.

FIG. 3 is another outward view of the exemplary therapeutic leg device shown in FIG. 1 illustrating exemplary coils and heating devices in dotted lines that are within the therapeutic leg device.

FIG. 4 is an exploded view of the exemplary therapeutic leg device shown in FIG. 1.

FIG. 5 is a front view of exemplary heating elements and vibration modules of the therapeutic device shown in FIG. 1.

FIG. 6A is a block diagram of exemplary circuitry of the therapeutic leg device shown in FIG. 1.

FIG. 6B is a block diagram of an exemplary batter providing power through relays to the heating elements and the vibration nodes.

FIG. 7 is a drawing showing the therapeutic leg device shown in FIG. 1 being used by an individual.

### DETAILED DESCRIPTION

The detailed description describes an exemplary therapeutic leg device in accordance with an embodiment of the present disclosure. The exemplary therapeutic leg device comprises an inward piece of material that is coupled to an outward piece of material. The inward piece of material is that piece of the therapeutic leg device that is adjacent the user's leg when the therapeutic leg device is worn by a user. Whereas, the outward piece of material is that piece of the therapeutic leg device that faces outwardly and is exposed when the therapeutic leg device is worn by the user. The therapeutic leg device wraps around the user's leg with the inward piece of material adjacent the user's leg and the outward piece of material faces outwardly. In one embodiment, the therapeutic leg device and is secured to the user's leg via a hook and loop fastener, often referred to as Velcro®.

Within an enclosure created by coupling the inward piece of material to the outward piece of material are therapeutic devices. In this regard, within the enclosure is a plurality of heating coils. The heating coils, when activated, emanate heat that is applied through the inward piece of material to the user's leg when the therapeutic leg device is worn. Additionally, within the enclosure is a plurality of massaging modules. The massaging modules provide vibration through the inward piece of material for massaging the user's leg when the therapeutic leg device is worn. The heating coils and the massaging modules may be activated separately or together via switches on the therapeutic leg device. Note that in one embodiment, the heating coils and the massaging modules are coupled to a time, and after a predetermined time, the switch may cut off the heating coils and the massaging modules.

Further, within the enclosure is an accelerometer. In one embodiment, the accelerometer senses motion in three dimensions, dimensions x, y, and z. Thus, when a user moves his/her leg, the accelerometer sends a signal to the switches that activate the heating coils and/or the massaging modules. Therefore, if a user is sleeping and the user moves his/her body, also moving the leg on which the therapeutic leg device is coupled, the switch is configured to activate the heating coils and/or the massaging modules.

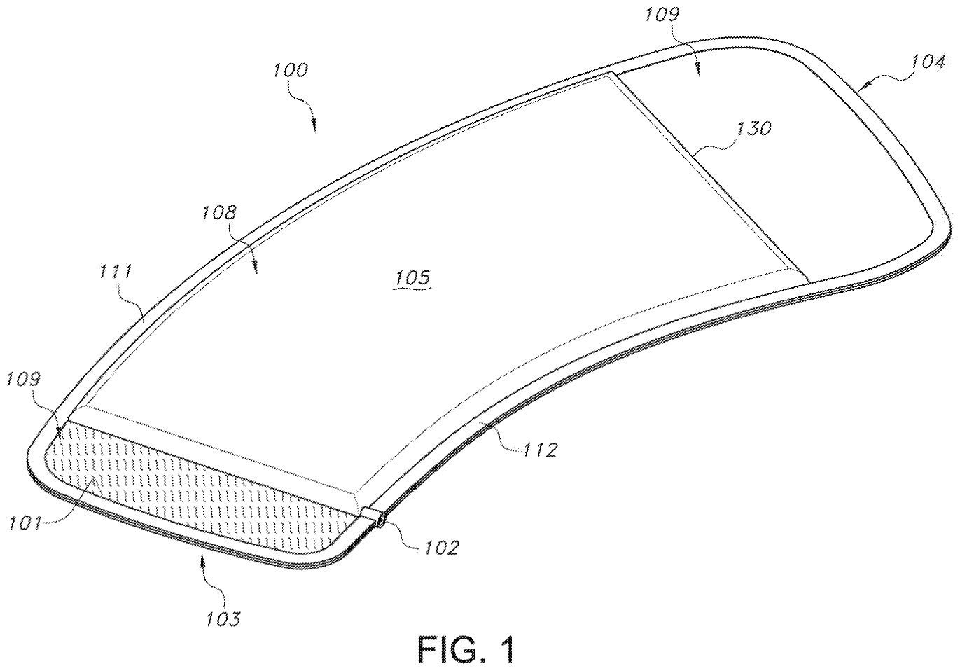

FIG. 1 is an interior covering 108 of a therapeutic leg massager 100 in accordance with an embodiment of the present disclosure. The interior covering 108 comprises a single piece of material 109 that extends the length of the interior covering 108. Further, the interior covering 108 comprises a first end 103 and a second end 104.

The interior covering 108 further comprises a central piece 105. The central piece 105 is coupled to sides 111,112 and is coupled at ends 130 and 132 to the piece of material 109. For example, the central piece 105 may be sewn to the sides 111, 112 and sewn at the ends 130 and 132 to the piece of material 109. The central piece 105 has a width that extends upward away from the material 109. Thus, the central piece 105 forms an interior cavity (not shown) on the underside of the interior covering 108. The cavity formed by the central piece 105 allows room for circuitry and components that perform functionality of the therapeutic leg massager 100.

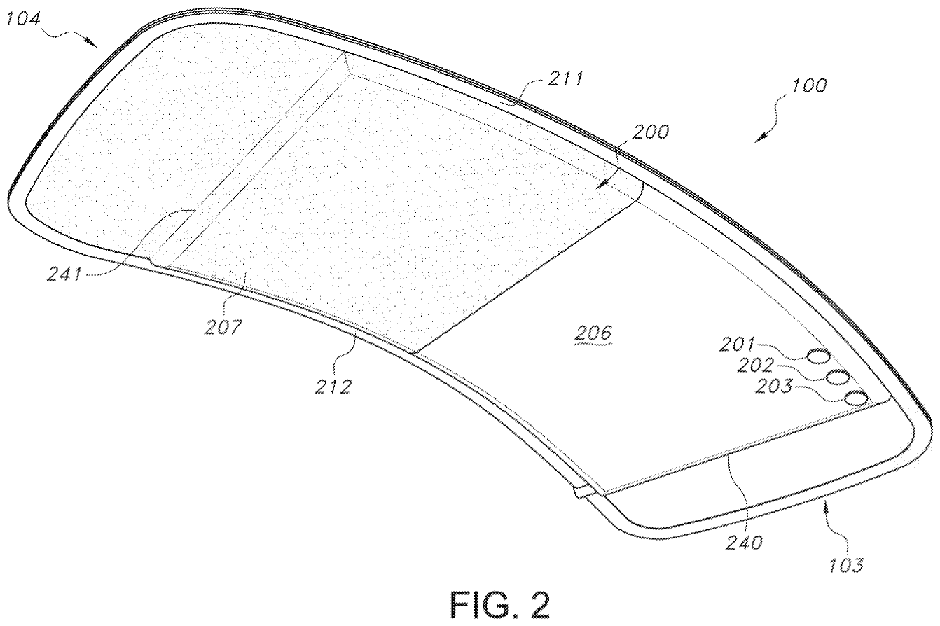

On the first end 103 of the interior covering 108 of the therapeutic leg massager 100 is a hook fastener 101 that couples to a loop fastener on an opposing side of the end 104. The loop fastener is described further with reference to FIG. 2. The hook fastener 101 mates with the loop fastener to secure the therapeutic leg massager 100 to a user's leg (not shown).

In the side 112 of the therapeutic leg massager 100 is a port 200. The port 200 is configured for received an end (not shown) of a cable (not shown). The cable is coupled to an alternating current/direct current (AC/DC) adapter and is used to charge the circuitry and components contained in the cavity formed by the central piece 105.

FIG. 2 is an exterior covering 200 of the exemplary therapeutic leg device 100 in accordance with an embodiment of the present disclosure. Note that the exterior covering 200 and the interior covering 108 are coupled together, e.g., sewn together. Note that other means of coupling the exterior covering 200 to the interior covering 108 may be used in other embodiments.

The exterior covering 200 comprises a piece of material 204 that extends the length of the exterior covering 200. The exterior covering 200 further comprises a central piece 206. The central piece 206 is coupled to sides 211, 212 and at ends 240 and 241 to the piece of material 204. For example, the central piece 206 may be sewn to the sides 211, 212 and sewn at ends 240 and 241 to the piece of material 204. The central piece 200 has a width that extends upward and away from the material 204. Thus, the central piece 206 forms an interior cavity (not shown) within the therapeutic leg massager 100. The interior cavity formed by the central piece 206 allows room for the circuitry and the components that perform functionality of the therapeutic leg massager 100.

Covering a portion of the material 204 at the end 104 and a portion of the central piece 206 is a loop fastener 207. Note that on the end 103 (FIG. 1) oldie interior covering 108 (FIG. 1) is hoop fastener 101 (FIG. 1). Thus, when the therapeutic leg massager 100 is wrapped around a user's leg with the interior covering 108 adjacent the user's leg, the hoop fastener 101 mates with the loop fastener 207 to secure the leg massager 100 to the user's leg, Further note that the tightness of the therapeutic leg massager may be adjusted by merely pulling the end 103 (FIG. 1) tighter over the piece of material 204 and the central piece 207 to connect the hoop fastener 101 to the loop fastener 107.

Note that the port 102 is also shown in FIG. 2. As described, the port 200 is configured for received an end (not shown) of a cable (not shown). The cable is coupled to an alternating current/direct current (AC/DC) adapter and is used to charge the circuitry and components contained in the cavity formed by the central piece 206.

The exterior covering 200 of the therapeutic leg massager 100 further comprises a plurality of buttons 201-203. A user may select button 201 to activate the vibration functionality of the therapeutic leg massager 100. The user may select button 202 to active the heating functionality of the therapeutic leg massager 100, and the user may select button 203 to activate both the healing and massaging functionality of the leg massager 100.

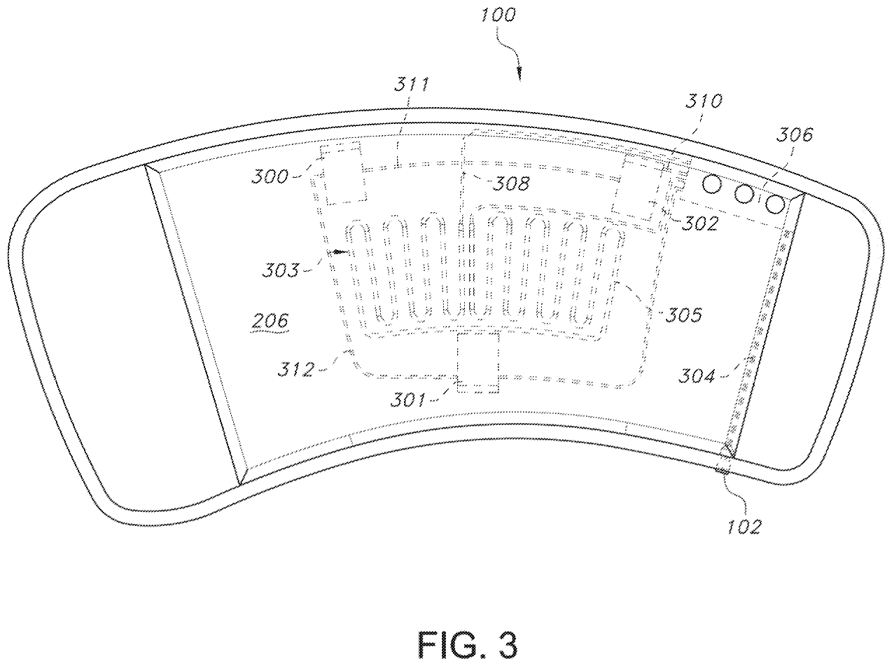

FIG. 3 is the interior covering 200 of the therapeutic leg massager 100 shown in FIG. 2. However, also depicted in FIG. 3 are the circuitry and components contained with the cavity formed by the central piece 105 (FIG. 1) and the central piece 206 (FIG. 2).

The cavity formed by the central piece 105 and the central piece 206 contains a plurality of heating elements 303 and 305. Note that in one embodiment the heating elements 303 and 305 are shown as single coils wherein each heating element 303 and 305 coils comprise a plurality of parallel coils that are integrally connected via corresponding arches. Note that other configurations are possible in other embodiments of the heating elements. The heating elements 303 and 305 are supplied power via connections 308 and 309, respectively, for heating by a switch 306, which is described further herein.

Further, the cavity formed by the central piece 105 and the central piece 206 contains a plurality of vibrating modules 300-302. When activated the modules 300-302 vibrate. Each node 300-302 is supplied power via connections 310-313. Note that in the embodiment depicted, the vibrating modules 300-302 are coupled to foe switch 306 serially such that when one node 300-302 vibrates, the remaining modules vibrate 300-302. Note that in other embodiments, the vibrating modules 300-302 may not be connected serially so that a user may be able to select one or a portion of the modules 300-302 to vibrate.

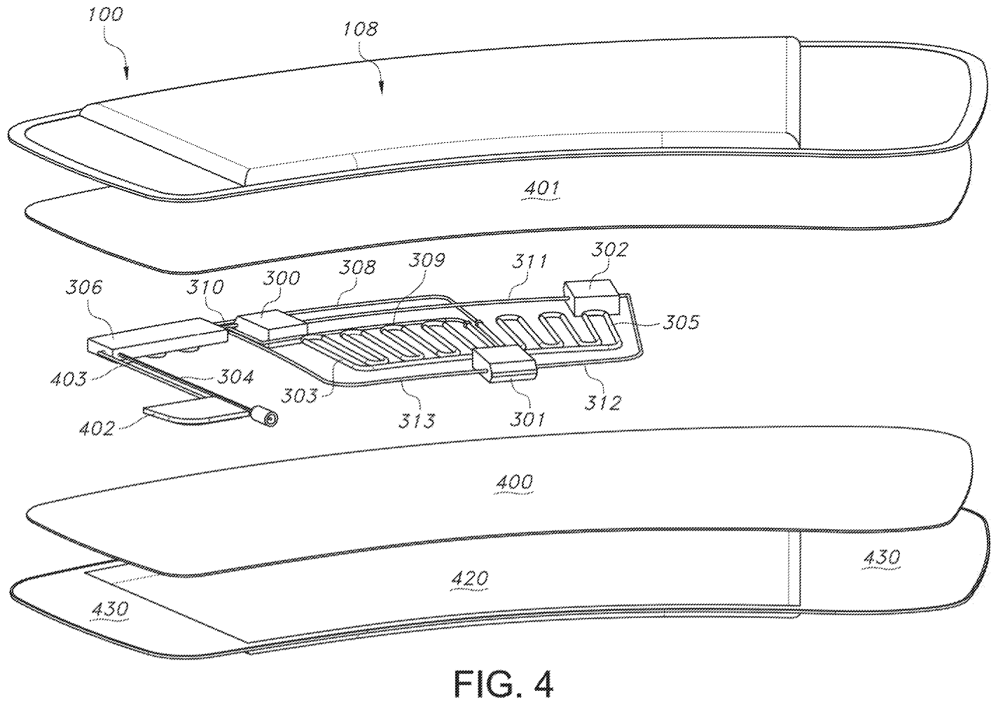

FIG. 4 is an exploded view of the therapeutic leg device 100 in accordance with an embodiment of the present disclosure. The therapeutic leg device 100 comprises the interior covering 108 and a corresponding pad 401 and the exterior covering 200 and a corresponding pad 400. Note that in one embodiment, the pads 400 and 401 may be made of foam; however, the pads 400 and 401 may be made of other materials in other embodiments.

Note that the exterior covering 200 comprises a cavity 420 formed by the central piece 206 (FIG. 2), which was described herein. Note that the interior covering 108 comprises the central piece 105 that also forms a cavity (not shown). The cavity formed by the exterior covering 200 and the interior covering (not shown) provide a cavity in which the circuitry and components may be housed.

The heating elements 303 and 305 are housed between the exterior covering 200 and the interior covering 108 in the respective cavities of the exterior covering 200 and the interior covering 108. The heating elements 303 and 305 are provided power via connections 309 and 308, respectively. In operation, power is provided to the heating elements 303 and 305, and the heating elements 303 and 305 heat up. Thus, the user wearing the therapeutic leg massager 100 may apply heat to his/her leg using the therapeutic leg massager 100.

Further, the vibrating modules 300-302 are housed in the respective cavities of the exterior covering 200 and the interior covering 108. The vibrating modules 300-302 are provided power via connections 310-313. In operation, power is provided to the vibrating modules 300-302, and the user wearing the therapeutic leg massager 100 may vibrate his/her leg.

Further the therapeutic leg massager 100 comprises an accelerometer 402. The accelerometer 402 detects motion in three dimensions. In operation, if the user falls asleep while wearing the therapeutic leg massager 100 and the user moves in any direction, the switch 306 is configured to turn on the heating elements 303 and 305 and/or the vibrating modules 300-302. Further, if the heating elements 303 and 305 and/or the vibrating modules 300-302 are on a timer so that the heating elements 303 and 305 and the vibrating modules 300-302 turn off during use, the user need only move his/her leg to restart the heating elements 303 and 305 and the vibrating modules 300-302.

Further shown in FIG. 4 is the charging port 102. Note that the charging port 102 may be used to change a battery in the switch 306. This battery power is then used to operate the accelerometer 402, the heating elements 303 and 305, and the vibrating modules 300-302.

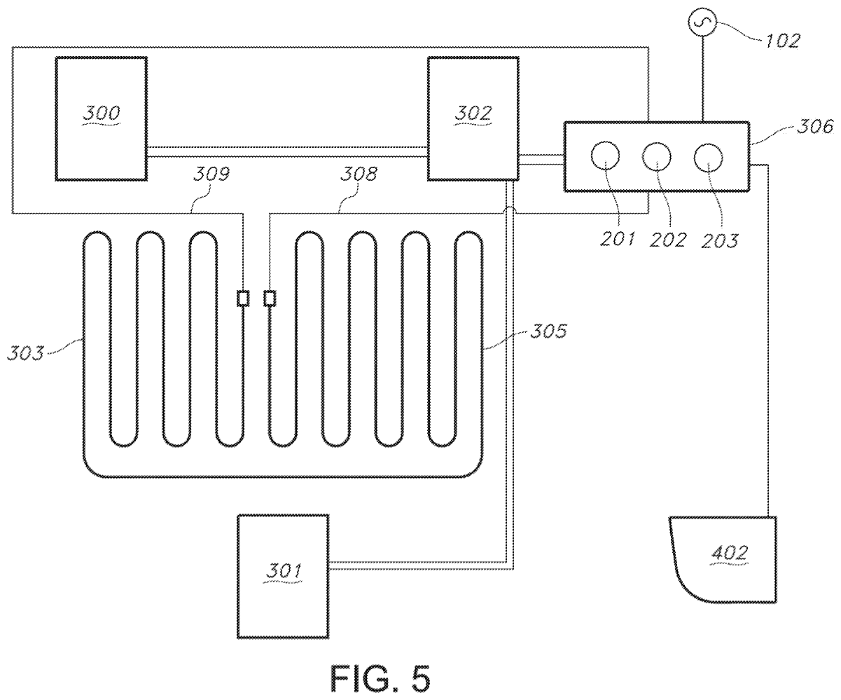

FIG. 5 shows exemplary configuration of the circuitry and components of the therapeutic leg massager 100.

The therapeutic leg massager 100 comprises the plurality of vibrating modules 300-302. As noted herein, button 201 may be selected by the user to activate the vibrating modules 300-302. In this regard, the user selects button 201, and the switch 306 provides power to the vibrating modules 300-302.

The therapeutic leg massager 100 comprises the plurality of heating elements 303 and 305. As noted herein, button 202 may be selected by the user to activate the heating elements 303-305. In this regard, the user selects button 202, and the switch 306 provides power to the heating elements 303 and 305.

The user may desire to use both the heating elements 303 and 305 and the vibrating modules 300-302 simultaneously. As noted herein, button 203 may be selected by the user to activate both the heating elements 303-305 and the vibrating modules 300-302. In this regard, the user selects button 203, and the switch 306 provides power to the heating elements 303 and 305 and to the vibrating modules 300-302.

Further, the therapeutic leg massager 100 comprises the accelerometer 402. Note that the accelerometer 402 is configured to detect motion in three dimensions, x, y, and z. Thus, if the user desires to activate the therapeutic leg massager 100 once the therapeutic leg massager 100 has gone in active, the user need merely move his/her leg. Upon detection of motion by the accelerometer 402, the switch 306 activates the heating elements 303 and 305 and the vibrating modules 300-302. Further, if the user falls asleep the accelerometer 402 may detect motions, e.g., the user rolls over in his/her sleep. Once this motion is detected, the switch 306 can activate the heating elements 303 and 305 and/or the vibration modules 300-302 while the user is asleep.

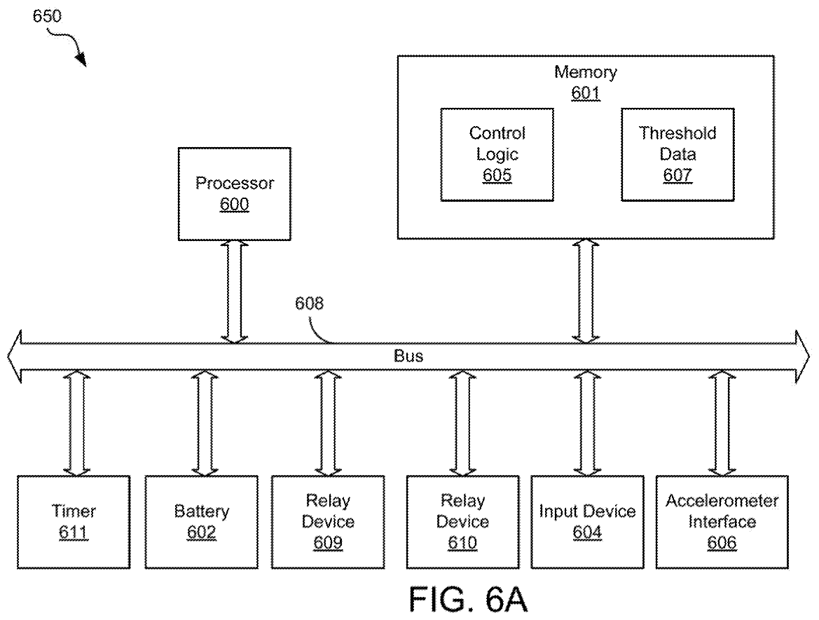

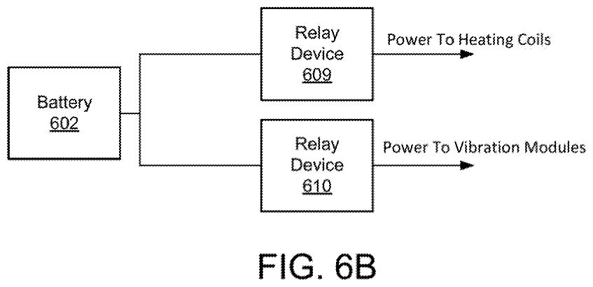

FIG. 6A is a block diagram of an exemplary circuitry 650 that may be implemented in switch 306 (FIG. 3) in accordance with an embodiment of the present disclosure. As shown by FIG. 6. the exemplary circuitry comprises a processor 600, a battery 602, an input device 604, two relays 609 and 610, a timer 611, and an accelerometer interface 606. Further, the circuitry comprises memory 601. Stored in memory 601 is control logic 605.

The control logic 605 generally controls the functionality of the therapeutic leg device 100, as will be described in more detail hereafter. It should be noted that the control logic 605 can be implemented in software, hardware, firmware or any combination thereof. In an exemplary embodiment illustrated in FIG. 6, the control logic 605 is implemented in software and stored in memory 601.

Note that the control logic 605, when implemented in software, can be stored and transported on any computer-readable medium for use by or in connection with an instruction execution apparatus that can fetch and execute instructions. In the context of this document, a “computer-readable medium” can be any means that can contain or store a computer program for use by or in connection with an instruction execution apparatus.

The exemplary embodiment of the circuitry 650 depicted by FIG. 6 comprises the at least one conventional processing element 600, such as a Digital Signal Processor (DSP) or a Central Processing Unit (CPU), that communicates to and drives the other elements within the circuitry 650 via a local interface 608, which can include at least one bus. Further, the processing element 601 is configured to execute instructions of software, such as the control logic 605.

The circuitry 650 further comprises threshold data 607 stored in memory 601. The threshold data is indicative of threshold values that if exceeded, the control logic will activate the relays 609 and 610 to provide power to the heating elements 303 and 305 and/or the vibration modules 300-302.

The input device 604 is any device for receiving input. In one embodiment, the input device 604 is the plurality of buttons 201,202, and 203 (FIG. 2). In such an embodiment button 201 activates the However, there me be other input devices in other embodiments.

The battery 602 is any type of battery known In the art or future-developed. In the present disclosure, the battery 602 is rechargeable, and when power is provided to port 102 (FIG. 1), the battery 602 recharges.

In operation, a user (not shown) may select the button 201. In response, the control logic 605 activates relay device 609 by closing the relay device 609, which establishes an electrical connection to the vibration modules 300-302 from the battery 602. If the user selects button 202, the control logic 605 activates relay device 610 by closing the relay device 610, which establishes an electrical connection from the battery 602 to the heating elements 303 and 305. Further, if the user selects button 203, the control logic 605 activates both relay device 609 and relay device 610 dosing both relay devices 609 and 610, which establishes an electrical connection to the vibration modules 300-302 and heating elements 303 and 305 from the battery 602.

In one embodiment, the control logic 605 may activate the timer 611. The timer 611 may be set to a threshold, for example, fifteen (15) minutes. Once the timer 611 meets the threshold time, the control logic 605 may deactivate the relays 609 and 610. This feature may be added as a safety precaution so that the user does not burn himself/herself by applying the heated heating elements 303 and 305 too long to his/her leg.

Further, the accelerometer interface 606 is any type of device for receiving data from the accelerometer 402 (FIG. 4). In operation, if the user moves higher leg or his/her body, the accelerometer 402 transmits data indicative of the movement to the accelerometer interface 606. In response, the control logic 605 compares the data indicative of the movement to the threshold data 607. If the comparison meets or exceeds the threshold data, the control logic 605 activates the relay device 609 and/or the relay device 610, which establishes and electrical connection to the vibration modules 300-302 and/or the heating elements 303 and 305.

FIG. 6A shows an exemplary connection of the battery 602 to the heating elements 303 and 305 and the vibration modules 300-302. Note that the battery 602 is coupled to the heating elements 303 and 305 through relay device 609. Further, the battery 602 is coupled to the vibration modules 300-302 through relay device 610. When the relay devices 609 and 610 are in an open position, no power is being provided to the vibration modules 300-302 or the heating elements 303 and 305.

As described above, if a user selects button 201 (FIG. 2), the control logic 605 (FIG. 6A) activates the relay device 610. Upon activation, power from the battery 602 is provided to the vibration modules 300-302 when the relay device 610 closes. Similarly, if a user selects button 202 (FIG. 2), the control logic 605 (FIG. 6A) activates the relay device 609. Upon activation, power from the battery 602 is provided to the heating elements 303 and 305 when the relay device 609 closes. Also, if a user selects button 203 (FIG. 2), the control logic 605 (FIG. 6A) activates the relay devices 609 and 610. Upon activation, power from the battery 602 is provided to both the heating elements 303 and 305 and the vibration modules 300-302 when the relay devices 609 and 610 close.

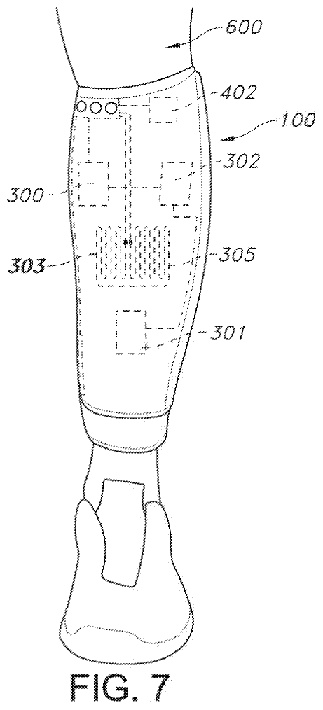

FIG. 7 shows the therapeutic leg massager 100 in use. In this regard, a user wraps the therapeutic leg massager 100 around his/her leg 600. The user may select the button 201 to start the vibration modules 300-302, select button 202 to start the heating elements 303 and 305, or the user may select button 203 to start both the vibration modules 300-302 and the heating elements 303 and 305.

If during use, the vibration modules 300-302 and or the heating elements 303 and 305 stop operating due to a preset time, the user can move his/her leg. The accelerometer 402 will detect the movement, and the switch will turn the heating elements 303 and 305 and the vibration modules 300-302 on. Note that the user may lay down and fall asleep, and the switch 306 may turn off the heating elements 303 and 305 and the vibration modules 300-302 off after a predetermined amount of time. In such a scenario, if the user moves his/her let or rolls over, the switch 306 will activate the heating elements 303 and 305 and the vibration modules 300-302 based upon input from the accelerometer.

What I claim is:

1. A leg massager, comprising:
an interior covering, the interior covering comprising a central raised section that extends from the exterior of the interior covering and forms a first cavity on an interior side of the interior covering;
an exterior covering, the exterior covering comprising a central raised section that extends from the exterior of the exterior covering a forms a second cavity on an interior side of the exterior covering;
one or more heating elements contained within the first and second cavities when the interior covering is coupled to the exterior covering; and
one or more vibration modules contained within the first and second cavities when the interior covering is coupled to the exterior covering.

2. The leg massager of claim 1, further comprising an accelerometer.

3. The leg massager of claim 2, further comprising a processor communicatively coupled to the accelerometer.

4. The leg massager of claim 3, wherein the processor is configured to activate the one or more heating elements when a value received from the accelerometer exceeds a threshold value.

5. The leg massager of claim 3, wherein the processor is configured to activate the one or more vibration modules when a value received from the accelerometer exceeds a threshold value.

6. The leg massager of claim 1, wherein a foam layer is inserted between the interior covering and the one or more heating elements and the one or more vibration modules.

7. The leg massager of claim 6, wherein a foam layer is inserted between the exterior covering and the one or more heating elements and the one or more vibration modules.

8. The leg massager of claim 1, further comprising a switch coupled to the exterior covering.

9. The leg massager of claim 8, wherein the switch comprises three buttons.

10. The leg massager of claim 9, wherein one of the three buttons controls the one or more heating elements.

11. The leg massager of claim 9, wherein on of the three buttons controls the one or more vibration modules.

12. The leg massager of claim 8, wherein one of the three buttons controls both the one or more heating elements and the one or more vibration modules.
